(19) United States
(12) Patent Application Publication
(10) Pub. No.: US 2025/0283163 A1
(43) Pub. Date: Sep. 11, 2025

(54) A CIRCUIT DESIGN TO MEASURE SMALL CURRENT VARIATION IN NANOPORE SEQUENCING

(71) Applicant: ILLUMINA, INC.

(72) Inventors: Xu LIU, Foster City, CA (US); Mohsen REZAEI, Brown Deer, WI (US); Arvin EMADI, Hayward, CA (US)

(21) Appl. No.: 18/575,205

(22) PCT Filed: Sep. 7, 2022
(86) PCT No.: PCT/US2022/076035

§ 371 Date: Dec. 28, 2023

Related U.S. Application Data
(60) Provisional application No. 63/261,056, filed on Sep. 9, 2021.

(51) Int. Cl. C12Q 1/6869; G01N 33/487

(52) U.S. Cl. CPC C12Q 1/6869; G01N 33/48721

(57) ABSTRACT

In one aspect, the disclosed technology relates to systems and methods for sequencing polynucleotides. In one embodiment, the disclosed system for sequencing polynucleotides includes: a sequencing cell comprising a nanopore for sensing a polynucleotide; an electronic circuit configured to measure an electrical response in the sequencing cell, the electronic circuit comprising a feedback capacitor; and a current source operably connected to the electronic circuit via a gate.

### CROSS-REFERENCE TO RELATED APPLICATIONS

This application is a U.S. national phase application under 35 U.S.C. §371 of International Application No. PCT/US2022/076035, filed on Sep. 7, 2022, which claims the benefit of U.S. Provisional Application No. 63/261,056, filed on Sep. 9, 2021, the disclosures of each of which are incorporated by reference in their entireties and for all purposes.

### REFERENCE TO SEQUENCE LISTING

The present application is being filed along with a Sequence Listing in electronic format. The Sequence Listing is provided as a file entitled “ILLINC.697NPSequenceListing.xml” created on Aug. 5, 2024, which is 2,361 bytes in size. The information in the electronic format of the Sequence Listing is incorporated herein by reference in its entirety.

### BACKGROUND

Some polynucleotide sequencing techniques involve performing a large number of controlled reactions on support surfaces or within predefined reaction chambers. The controlled reactions may then be observed or detected, and subsequent analysis may help identify properties of the polynucleotide involved in the reaction. Examples of such sequencing techniques include next-generation sequencing or massive parallel sequencing involving sequencing-by-ligation, sequencing-by-synthesis, reversible terminator chemistry, or pyrosequencing approaches.

Some polynucleotide sequencing techniques utilize a nanopore, which can provide a path for an ionic electrical current. For example, as the polynucleotide traverses through the nanopore, it influences the electrical current through the nanopore. Each passing nucleotide, or series of nucleotides, that passes through the nanopore yields a characteristic electrical current. These characteristic electrical currents of the traversing polynucleotide can be recorded to determine the sequence of the polynucleotide.

### SUMMARY

Provided in examples herein are methods for sequencing biopolymers, particularly polynucleotides, and systems and kits for performing the methods.

The systems, devices, kits, and methods disclosed herein each have several aspects, no single one of which is solely responsible for their desirable attributes. Without limiting the scope of the claims, some prominent features will now be discussed briefly. Numerous other examples are also contemplated, including examples that have fewer, additional, and/or different components, steps, features, objects, benefits, and advantages. The components, aspects, and steps may also be arranged and ordered differently. After considering this discussion, and particularly after reading the section entitled “Detailed Description,” one will understand how the features of the devices and methods disclosed herein provide advantages over other known devices and methods.

In some applications of nanopore nucleic acid sequencing, there is a need for measuring small electrical current variations (e.g., in the pA range). Commonly used current measuring techniques and devices, e.g., analog-to digital converters (ADCs), may not have sufficient sensitivity to accurately measure such small electrical current variations. Therefore, measuring techniques or devices that can dynamically adjust the analog-to digital region are needed.

In some embodiments, disclosed is a circuit design that can dynamically adjust the analog-to digital region to only convert the current range that matter the most. In some embodiments, disclosed is a switch that can switch between an item to be measured (e.g., the nanopore) and a current source. During each measuring cycle, the switch first connects to the current source such that the current source pre-charges a feedback capacitor to a negative value. Then, the switch connects to the item to be measured to obtain a signal. Compared to an actual value of signal, the measured signal value can be shifted by a DC value from the current source. This way, an AC part of the signal can be extracted. The measured signal value may be fed to an ADC, and the ADC will only need to convert the AC part of the signal.

Additional details of exemplary nanopore sequencing devices and methods of operating the devices that can be used in conjunction with the present disclosure can be found in U.S. Provisional Patent Application Nos. 63/200,868 and 63/169,041 (International Patent Application Numbers PCT/US2021/038125 and PCT/US2022/020395), the entirety of each of the disclosures is incorporated herein by reference.

It is to be understood that any features of the device and/or of the array disclosed herein may be combined together in any desirable manner and/or configuration. Further, it is to be understood that any features of the method of using the device may be combined together in any desirable manner. Moreover, it is to be understood that any combination of features of this method and/or of the device and/or of the array may be used together, and/or may be combined with any of the examples disclosed herein. Still further, it is to be understood that any feature or combination of features of any of the devices and/or of the arrays and/or of any of the methods may be combined together in any desirable manner, and/or may be combined with any of the examples disclosed herein.

It should be appreciated that all combinations of the foregoing concepts and additional concepts discussed in greater detail below are contemplated as being part of the inventive subject matter disclosed herein and may be used to achieve the benefits and advantages described herein.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A schematically illustrates an example of DNA translocation through a solid-state nanopore.

FIG. 1B schematically illustrates an example of DNA translocation through a protein nanopore.

FIG. 2 schematically illustrates an example integration of nanopore array with a readout integrated circuit (ROIC).

FIG. 3 schematically illustrates one of the nanopore unit cells shown in FIG. 2.

FIG. 4 schematically illustrates an equivalent circuit of the unit cell shown in FIG. 3.

FIG. 5 schematically illustrates electrical current variations at a nanopore.

FIG. 6 schematically illustrates an example circuit design for measuring small current variation.

### DETAILED DESCRIPTION

All patents, applications, published applications and other publications referred to herein are incorporated herein by reference to the referenced material and in their entireties. If a term or phrase is used herein in a way that is contrary to or otherwise inconsistent with a definition set forth in the patents, applications, published applications and other publications that are herein incorporated by reference, the use herein prevails over the definition that is incorporated herein by reference.

### Definitions

All technical and scientific terms used herein have the same meaning as commonly understood to one of ordinary skill in the art to which this disclosure belongs unless clearly indicated otherwise.

As used herein, the singular forms “a”, “and”, and “the” include plural referents unless the context clearly dictates otherwise. Thus, for example, reference to “a sequence” may include a plurality of such sequences, and so forth.

The terms comprising, including, containing and various forms of these terms are synonymous with each other and are meant to be equally broad. Moreover, unless explicitly stated to the contrary, examples comprising, including, or having an element or a plurality of elements having a particular property may include additional elements, whether or not the additional elements have that property.

As used herein, the term “operably connected” refers to a configuration of elements, wherein an action or reaction of one element affects another element, but in a manner that preserves each element's functionality.

As used herein, the term “membrane” refers to a non-permeable or semi-permeable barrier or other sheet that separates two liquid/gel chambers (e.g., a cis well and a fluidic cavity) which can contain the same compositions or different compositions therein. The permeability of the membrane to any given species depends upon the nature of the membrane. In some examples, the membrane may be non-permeable to ions, to electric current, and/or to fluids. For example, a lipid membrane may be impermeable to ions (i.e., does not allow any ion transport therethrough), but may be at least partially permeable to water (e.g., water diffusivity ranges from about 40 μm/s to about 100 μm/s). For another example, a synthetic/solid-state membrane, one example of which is silicon nitride, may be impermeable to ions, electric charge, and fluids (i.e., the diffusion of all of these species is zero). Any membrane may be used in accordance with the present disclosure, as long as the membrane can include a transmembrane nanoscale opening and can maintain a potential difference across the membrane. The membrane may be a monolayer or a multilayer membrane. A multilayer membrane includes two or more layers, each of which is a non-permeable or semi-permeable material.

The membrane may be formed of materials of biological or non-biological origin. A material that is of biological origin refers to material derived from or isolated from a biological environment, such as an organism or cell, or a synthetically manufactured version of a biologically available structure (e.g., a biomimetic material).

An example membrane that is made from the material of biological origin includes a monolayer formed by a bolalipid. Another example membrane that is made from the material of biological origin includes a lipid bilayer. Suitable lipid bilayers include, for example, a membrane of a cell, a membrane of an organelle, a liposome, a planar lipid bilayer, and a supported lipid bilayer. A lipid bilayer can be formed, for example, from two opposing layers of phospholipids, which are arranged such that their hydrophobic tail groups face towards each other to form a hydrophobic interior, whereas the hydrophilic head groups of the lipids face outwards towards the aqueous environment on each side of the bilayer. Lipid bilayers can also be formed, for example, by a method in which a lipid monolayer is carried on an aqueous solution/air interface past either side of an aperture that is substantially perpendicular to that interface. The lipid is normally added to the surface of an aqueous electrolyte solution by first dissolving it in an organic solvent and then allowing a drop of the solvent to evaporate on the surface of the aqueous solution on either side of the aperture. Once the organic solvent has at least partially evaporated, the solution/air interfaces on either side of the aperture are physically moved up and down past the aperture until a bilayer is formed. Other suitable methods of bilayer formation include tip-dipping, painting bilayers, and patch-clamping of liposome bilayers. Any other methods for obtaining or generating lipid bilayers may also be used.

A material that is not of biological origin may also be used as the membrane. Some of these materials are solid-state materials and can form a solid-state membrane, and others of these materials can form a thin liquid film or membrane. The solid-state membrane can be a monolayer, such as a coating or film on a supporting substrate (i.e., a solid support), or a freestanding element. The solid-state membrane can also be a composite of multilayered materials in a sandwich configuration. Any material not of biological origin may be used, as long as the resulting membrane can include a transmembrane nanoscale opening and can maintain a potential difference across the membrane. The membranes may include organic materials, inorganic materials, or both. Examples of suitable solid-state materials include, for example, microelectronic materials, insulating materials (e.g., silicon nitride (Si₃N₄), aluminum oxide (Al₂O₃), hafnium oxide (HfO₂), tantalum pentoxide (Ta₂O₅), silicon oxide (SiO₂), etc.), some organic and inorganic polymers (e.g., polyamide, plastics, such as polytetrafluoroethylene (PTFE), or elastomers, such as two-component addition-cure silicone rubber), and glasses. In addition, the solid-state membrane can be made from a monolayer of graphene, which is an atomically thin sheet of carbon atoms densely packed into a two-dimensional honeycomb lattice, a multilayer of graphene, or one or more layers of graphene mixed with one or more layers of other solid-state materials. A graphene-containing solid-state membrane can include at least one graphene layer that is a graphene nanoribbon or graphene nanogap, which can be used as an electrical sensor to characterize the target polynucleotide. It is to be understood that the solid-state membrane can be made by any suitable method, for example, chemical vapor deposition (CVD). In an example, a graphene membrane can be prepared through either CVD or exfoliation from graphite. Examples of suitable thin liquid film materials that may be used include diblock copolymers or triblock copolymers, such as amphiphilic PMOXA-PDMS-PMOXA ABA triblock copolymers.

As used herein, the term “nanopore” is intended to mean a hollow structure discrete from, or defined in, and extending across the membrane. The nanopore permits ions, electric current, and/or fluids to cross from one side of the membrane to the other side of the membrane. For example, a membrane that inhibits the passage of ions or water-soluble molecules can include a nanopore structure that extends across the membrane to permit the passage (through a nanoscale opening extending through the nanopore structure) of the ions or water-soluble molecules from one side of the membrane to the other side of the membrane. The diameter of the nanoscale opening extending through the nanopore structure can vary along its length (i.e., from one side of the membrane to the other side of the membrane), but at any point is on the nanoscale (i.e., from about 1 nm to about 100 nm, or to less than 1000 nm). Examples of the nanopore include, for example, biological nanopores, solid-state nanopores, and biological and solid-state hybrid nanopores. In some embodiments, a nanopore refers to a pore having an opening with a diameter at its most narrow point of about 0.3 nm to about 2 nm. For example, a nanopore may be a solid-state nanopore, a graphene nanopore, an elastomer nanopore, or may be a naturally-occurring or recombinant protein that forms a tunnel upon insertion into a bilayer, thin film, membrane, or solid-state aperture, also referred to as a protein pore or protein nanopore herein (e.g., a transmembrane pore). If the protein inserts into the membrane, then the protein is a tunnel-forming protein.

As used herein, the term “biological nanopore” is intended to mean a nanopore whose structure portion is made from materials of biological origin. Biological origin refers to a material derived from or isolated from a biological environment such as an organism or cell, or a synthetically manufactured version of a biologically available structure. Biological nanopores include, for example, polypeptide nanopores and polynucleotide nanopores.

As used herein, the term “polypeptide nanopore” is intended to mean a protein/polypeptide that extends across the membrane, and permits ions, electric current, polymers such as DNA or peptides, or other molecules of appropriate dimension and charge, and/or fluids to flow therethrough from one side of the membrane to the other side of the membrane. A polypeptide nanopore can be a monomer, a homopolymer, or a heteropolymer. Structures of polypeptide nanopores include, for example, an α-helix bundle nanopore and a β-barrel nanopore. Example polypeptide nanopores include α-hemolysin, Mycobacterium smegmatis porin A (MspA), gramicidin A, maltoporin, OmpF, OmpC, PhoE, Tsx, F-pilus, acrolysin, etc. The protein α-hemolysin is found naturally in cell membranes, where it acts as a pore for ions or molecules to be transported in and out of cells. Mycobacterium smegmatis porin A (MspA) is a membrane porin produced by Mycobacteria, which allows hydrophilic molecules to enter the bacterium. MspA forms a tightly interconnected octamer and transmembrane beta-barrel that resembles a goblet and contains a central pore.

A polypeptide nanopore can be synthetic. A synthetic polypeptide nanopore includes a protein-like amino acid sequence that does not occur in nature. The protein-like amino acid sequence may include some of the amino acids that are known to exist but do not form the basis of proteins (i.e., non-proteinogenic amino acids). The protein-like amino acid sequence may be artificially synthesized rather than expressed in an organism and then purified/isolated.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof such as locked nucleic acids (LNA) and peptide nucleic acids (PNA). A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA, LNA, or PNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, the term “solid-state nanopore” is intended to mean a nanopore whose structure portion is defined by a solid-state membrane and includes materials of non-biological origin (i.e., not of biological origin). A solid-state nanopore can be formed of an inorganic or organic material. Solid-state nanopores include, for example, silicon nitride nanopores, silicon dioxide nanopores, and graphene nanopores.

The nanopores disclosed herein may be hybrid nanopores. A “hybrid nanopore” refers to a nanopore including materials of both biological and non-biological origins. An example of a hybrid nanopore includes a polypeptide-solid-state hybrid nanopore and a polynucleotide-solid-state nanopore.

The application of the potential difference across a nanopore may force the translocation of a nucleic acid through the nanopore. One or more signals are generated that correspond to the translocation of the nucleotide through the nanopore. Accordingly, as a target polynucleotide, or as a mononucleotide or a probe derived from the target polynucleotide or mononucleotide, transits through the nanopore, the current across the membrane changes due to base-dependent (or probe dependent) blockage of the nanopore constriction, for example. The signal from that change in current can be measured using any of a variety of methods. Each signal is unique to the species of nucleotide(s) (or probe) in the nanopore, such that the resultant signal can be used to determine a characteristic of the polynucleotide. For example, the identity of one or more species of nucleotide(s) (or probe) that produces a characteristic signal can be determined.

As used herein, the term “nanopore sequencer” refers to any of the devices disclosed herein that can be used for nanopore sequencing. In the examples disclosed herein, during nanopore sequencing, the nanopore is immersed in examples of the electrolyte disclosed herein and a potential difference is applied across the membrane. In an example, the potential difference is an electric potential difference or an electrochemical potential difference. An electrical potential difference can be imposed across the membrane via a voltage source that injects or administers current to at least one of the ions of the electrolyte contained in the cis well or one or more of the trans wells. An electrochemical potential difference can be established by a difference in ionic composition of the cis and trans wells in combination with an electrical potential. The different ionic composition can be, for example, different ions in each well or different concentrations of the same ions in each well.

As used herein, a “nucleotide” includes a nitrogen containing heterocyclic base, a sugar, and one or more phosphate groups. Nucleotides are monomeric units of a nucleic acid sequence. Examples of nucleotides include, for example, ribonucleotides or deoxyribonucleotides. In ribonucleotides (RNA), the sugar is a ribose, and in deoxyribonucleotides (DNA), the sugar is a deoxyribose, i.e., a sugar lacking a hydroxyl group that is present at the 2′ position in ribose. The nitrogen containing heterocyclic base can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. The phosphate groups may be in the mono-, di-, or tri-phosphate form. These nucleotides are natural nucleotides, but it is to be further understood that non-natural nucleotides, modified nucleotides or analogs of the aforementioned nucleotides can also be used.

As used herein, the term “signal” is intended to mean an indicator that represents information. Signals include, for example, an electrical signal and an optical signal. The term “electrical signal” refers to an indicator of an electrical quality that represents information. The indicator can be, for example, current, voltage, tunneling, resistance, potential, voltage, conductance, or a transverse electrical effect. An “electronic current” or “electric current” refers to a flow of electric charge. In an example, an electrical signal may be an electric current passing through a nanopore, and the electric current may flow when an electric potential difference is applied across the nanopore.

The aspects and examples set forth herein and recited in the claims can be understood in view of the above definitions.

### Sequencing Using a Nanopore

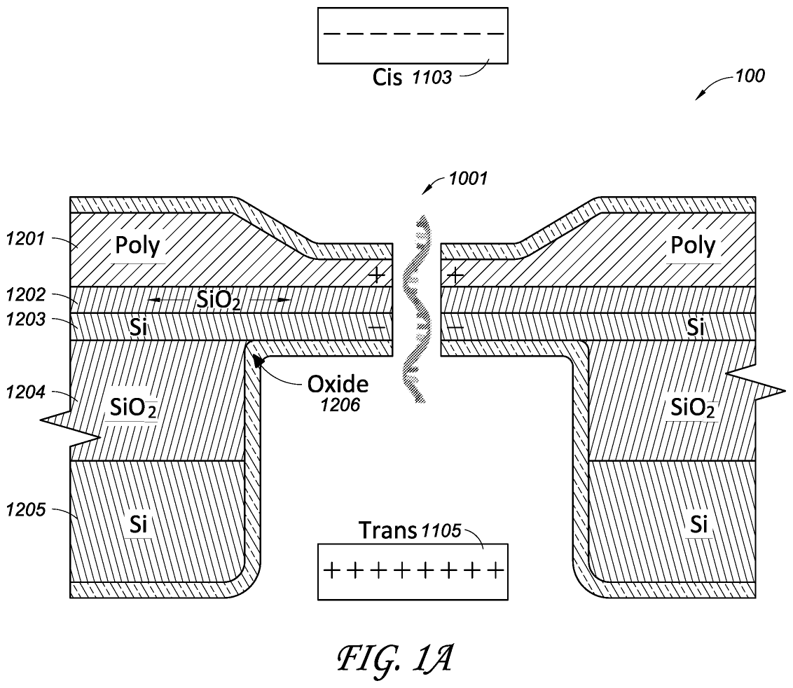

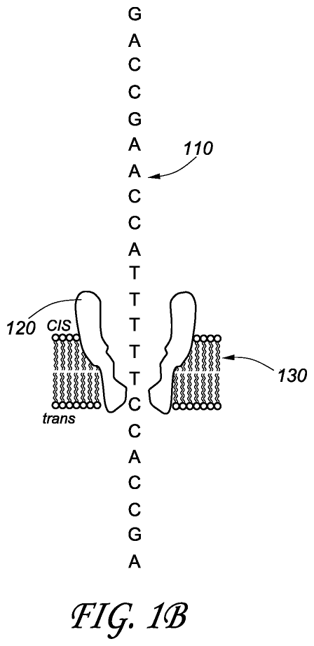

Polynucleotides may be sequenced using a nanopore unit cell, or a nanopore sensor, based on electrical responses. In some embodiments, such unit cell may include a nanopore, a flow chamber containing a liquid, one or more electrodes, and an electronic circuit for measurement. In some cases, the nanopore may be a solid-state nanopore as illustrated in FIG. 1A. In some cases, the nanopore may be a solid-state nanopore directly formed as a nanoscale opening in a membrane (e.g., silicon based, graphene, or polymer membrane). A polynucleotide may be dissolved in the liquid, e.g., an electrolyte. In some embodiments, application of a voltage via the one or more electrodes results in a driving force and/or a change in the electrical conditions that are suitable for driving translocation of the polynucleotide through the nanopore, for example from the “cis” side to the “trans” side, or vice versa. As the polynucleotide translocates through the nanopore, the polynucleotide may modulate the electrical properties of the nanopore, such that the nucleobase sequence of the polynucleotide can be identified. For example, the electrical current through the nanopore or the electrical resistance at the nanopore may be a function of the identity of the nucleobase of the polynucleotide at or near the nanopore. FIG. 1A schematically illustrates an example of a polynucleotide 1001 translocating through a solid-state nanopore device 100. The solid-state nanopore device 100 includes a silicon substrate 1205; a silicon dioxide layer 1204 formed on the silicon substrate 1205; and a stack of polysilicon 1201, silicon dioxide 1202 and silicon 1203 materials formed on the silicon dioxide layer 1204. A silicon oxide layer 1206 may be grown on the surfaces of the device 100 and may insulate the device 100. A nano-scale opening is formed in the stack of polysilicon 1201, silicon dioxide 1202 and silicon 1203 materials, allowing the polynucleotide 1001 to pass through. The device 100 may further include a cis electrode 1103 and a trans electrode 1105 for application of a voltage across the device 100. An electrolyte may be filled in the chambers between the electrodes 1103 and 1105 and the silicon oxide layer 1206. The polynucleotide 1001 may be negatively charged in the electrolyte and may thus be driven through the nano-scale opening from the cis side to the trans side or vice versa when a voltage difference between the cis electrode 1103 and the trans electrode 1105 is applied.

In some cases, the nanopore may be a biological nanopore formed of peptides or polynucleotides and deposited in a lipid bilayer or a polymer membrane, e.g., a synthetic polymeric membrane. In an example shown in FIG. 1B, a protein nanopore 120 is deposited in a lipid bilayer 130. A single-stranded DNA 110 is passing, from the “cis” side, through the nanopore 120, to the “trans” side, or vice versa. Applying a voltage across the “cis” side to the “trans” side results in an ionic current through the nanopore. When a nucleotide of the DNA 110 is in or near the nanopore, it may result in a unique ionic current blockade at the nanopore 120, and therefore a unique nanopore resistance depending on the identity of the nucleotide. By measuring the ionic current or the nanopore resistance, the nucleotide at or near the nanopore can be identified.

In other embodiments, the DNA 110 may not pass through the nanopore 120. A unique tag or label for a nucleotide in the DNA 110 may pass through the nanopore 120. In one example, a tag or label of the nucleotide may be a particular sequence combination of nucleotides. When the tag or label is in or near the nanopore, it may result in a unique ionic current blockade at the nanopore, and therefore a unique nanopore resistance depending on the identity of the molecule of interest. By measuring the ionic current or the nanopore resistance, the tag or label at or near the nanopore, and therefore the corresponding nucleotide, can be identified.

Although embodiments herein describe determining a signal level by determining the ionic current through the nanopore, embodiments also include alone or in combination determining the signal level by measuring other electrical characteristics of the cis/trans nanopore cell. For example, in other embodiments, a signal level is determined by the voltage potential at a specified area or component of the cis/trans nanopore cell. For example, in other embodiments, a signal level is determined by the electrical impedance at a specified area or component of the cis/trans nanopore cell. For example, in other embodiments, a signal level is determined by the conductivity/resistance of the nanopore membrane.

In other embodiments, sequencing of a target polynucleotide may involve nanopore sensing of (1) a single-stranded portion of the target polynucleotide; (2) a nucleic acid duplex of a portion of the target polynucleotide; (3) a label or tag that can be tethered or untethered to the target polynucleotide; or any combination thereof.

In some embodiments, multiple such nanopore unit cells may be arranged in an array, and each unit cell or each nanopore sensor may be individually accessed by a logic circuit.

### Measurement Circuit for Nanopore Sequencing

In some embodiments, a nanopore array is formed of an array of biochemical sensors, e.g., an array of nanopore unit cells described above. In some embodiments, a nanopore array can be used to perform long read DNA sequencing. A characteristic feature of a nanopore array is G-base per second per square centimeter of a chip. In some embodiments, to achieve higher data rate, the density of nanopores in a 2D array is increased. In some embodiments, a 2D readout circuit is used to take measurements from a 2D nanopore array.

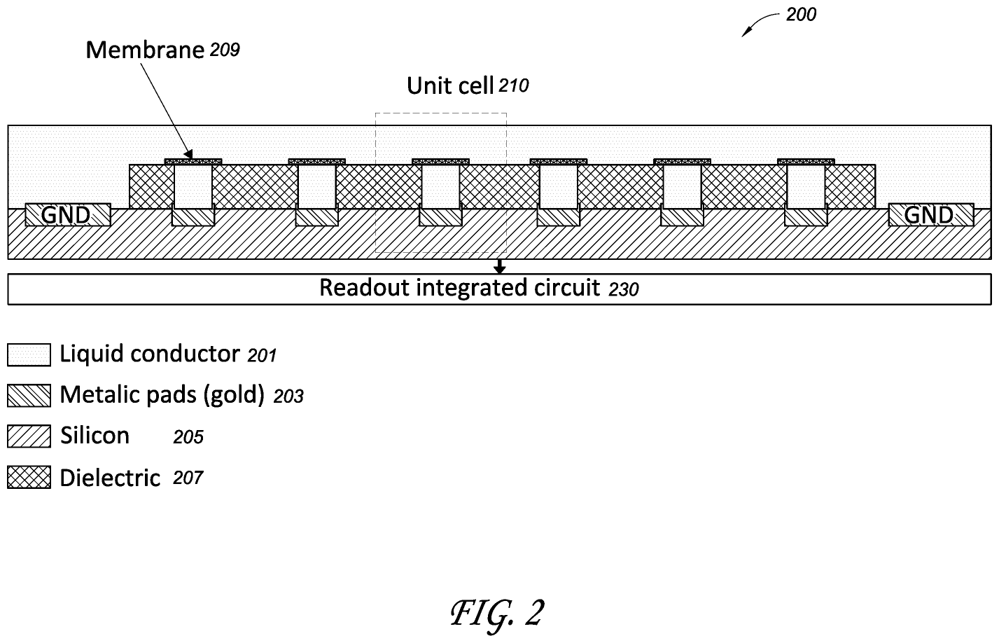

FIG. 2 schematically illustrates an example integration of nanopore array 200 with a readout integrated circuit (ROIC). In one example, the nanopore array 200 is formed on a silicon substrate 205, which is integrated with a CMOS readout integrated circuit 230. In one example, the nanopore array 200 includes a plurality of nanopore unit cells 210. Individual nanopore unit cells 210 may be separated by dielectric 207. In one example, the nanopore array 200 may be a 2D high density nanopore array. In some embodiments, each nanopore unit cell 210 is operably connected to a common ground via an electrode (e.g., a metallic pad 203 shown in FIG. 2) in the unit cell 210. In some embodiments, each nanopore unit cell 210 contains a conductor liquid 201 and a membrane 209 for inserting a nanopore. Each nanopore unit cell 210 may include a nanopore shown and described in connection with FIG. 1A or FIG. 1B.

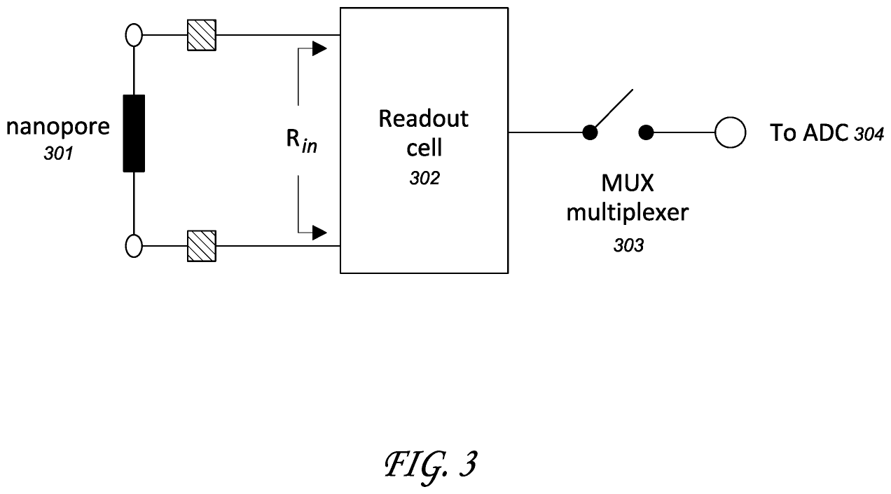

FIG. 3 schematically illustrates one of the nanopore unit cells shown in FIG. 2. Each nanopore unit cell reads data from a nanopore 301 using a corresponding readout cell 302. The readout cell 302 may be coupled to a multiplexer (MUX) 303, which may be coupled to an analog-to-digital converter (ADC) 304. The readout cell 302 may have an equivalent input resistance Rᵢₙ.

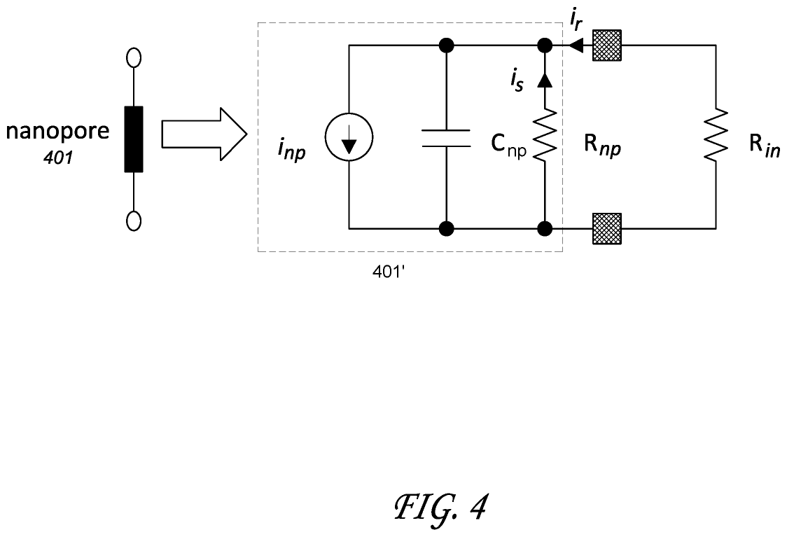

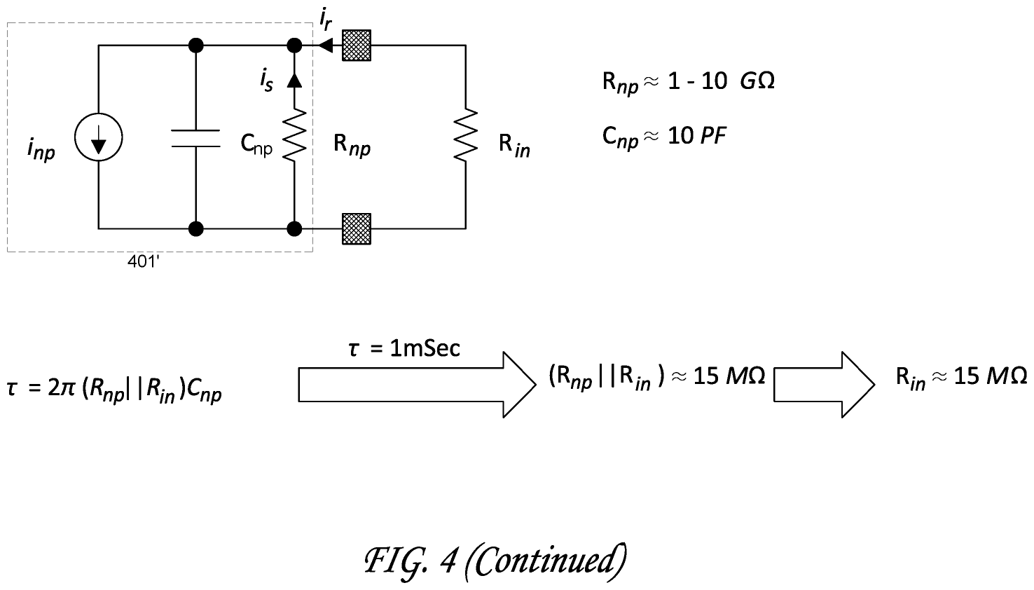

FIG. 4 schematically illustrates an equivalent circuit of the unit cell shown in FIG. 3. A nanopore 401 may have an equivalent circuit diagram 401′ that includes a current source iᵤₚ, a capacitance Cᵤₚ and a resistance Rᵤₚ. In a preferred embodiment, input resistance of the readout cell Rᵢₙ is small enough to allow a fast readout. In one example, if Rᵢₙ<<Rᵤₚ, unit cell response time may be determined by the input resistance of the readout cell. Example values of resistances, capacitance and circuit time constant are shown in FIG. 4 (Continued).

### Increasing the Digitization Efficiency

Some applications of nanopore sequencing involve sensing electrical currents over a large range of values (e.g., 0-1 nA), for example when the polynucleotide is not in the nanopore versus when the polynucleotide is in the nanopore. However, when the polynucleotide is translocating through the nanopore and different nucleobases are at or near the nanopore constriction, electrical current variations may be comparatively small (e.g., in the pA range).

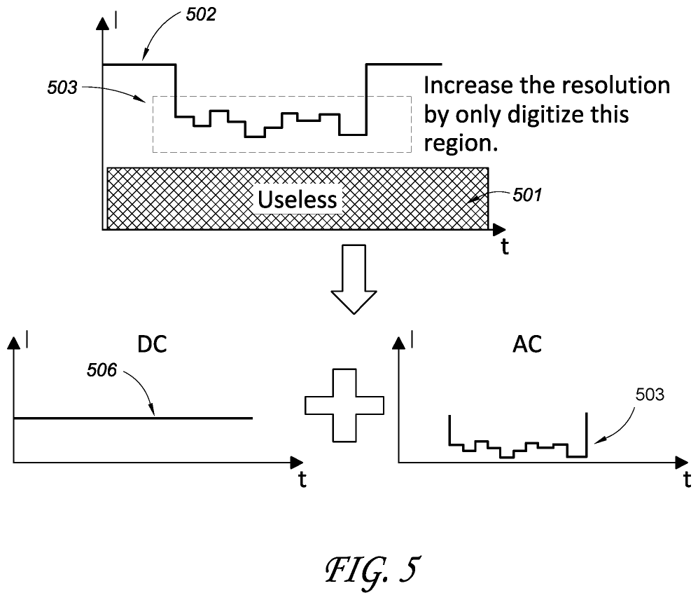

For example, FIG. 5 illustrates the electrical current 502 as a function of time at a nanopore. The baseline level 501 of the electrical current 502 may not include useful information for identifying nucleotide sequence of the polynucleotide. As shown in FIG. 5, the electrical current 502 at a nanopore may be decomposed into a DC (direct current) component 506 and an AC (alternating current) component 503. The AC component 503 may represent small electrical current variations, e.g., in the 0.1 to 100 pA range, when different nucleobases, different single-stranded portions of a target polynucleotide, different duplex portions of a target polynucleotide, and/or different labels are present at or near the nanopore constriction. Digitizing the AC component 503 may increase the readout resolution of the nanopore unit cell. The DC component 506 may represent large electrical current variations, e.g., in the 0-1 nA range, when a target polynucleotide is being sequenced by the nanopore versus when no polynucleotide is being sequenced by the nanopore. In some embodiments, to be able to accurately perform sequencing of a target polynucleotide, an electrical current resolution of the AC component in the 0.1 to 100 pA range may be needed, while the overcall electrical current operation range of a nanopore unit cell may be about 0-1 nA. In some embodiments, to be able to accurately perform sequencing of a target polynucleotide, a temporal resolution of the AC component in the 0.1 milliseconds to 100 seconds range may be needed.

In some embodiments, dynamically adjusting the analog-to-digital range of a readout cell can increase the readout resolution of a nanopore unit cell. For example, as illustrated in FIG. 5, the DC component of the electrical current may be removed, and only the AC component may be digitized and read-out. In other examples, the AC component may be filtered or isolated from the DC component and amplified.

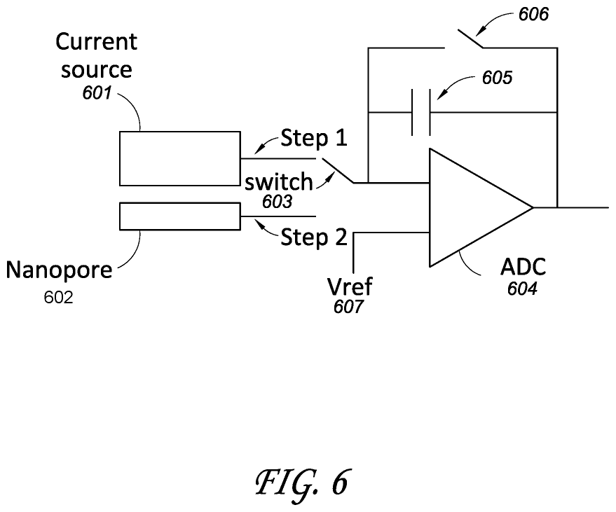

In some embodiments, a readout cell may include an example readout circuit design for measuring small current variation as illustrated in FIG. 6. The readout circuit design shown in FIG. 6 may allow a current resolution of an AC component in the 0.1 to 100 pA range out of an overcall, 0-1 nA operation range of a nanopore unit cell. The readout circuit design shown in FIG. 6 may allow a temporal resolution of an AC component in the 0.1 milliseconds to 100 seconds range. The readout circuit may include an analog-to-digital converter (ADC) 604 operably connected with a capacitor 605 to create a feedback loop. The feedback capacitor may be connected to one of the input terminals and the output terminal of the ADC 604. In some examples, the capacitor 605 may be connected in parallel with a switch 606. The readout circuit may include a switch/gate 603 that may either connect to a current source 601 via “step 1” or to a nanopore sequencing unit cell 602 via “step 2”. The switch/gate 603 may be connected to one of the input terminals of the ADC 604. A reference voltage 607, Vref, may be supplied to the other one of the input terminals of the ADC 604. The nanopore sequencing unit cell 602 may include a nanopore for sensing a polynucleotide, for example a nanopore shown in FIG. 1A or FIG. 1B. During polynucleotide sequencing, the capacitor 605 may be pre-charged in each sampling cycle to shift down the baseline of the ADC 604. In some embodiments, the switch/gate 603 or the switch 606 may be a solid-state switch, such as a transistor, e.g., a metal-oxide-semiconductor field-effect transistor (MOSFET).

In an example sequencing operation process, each sampling cycle include:
1. Connect the switch/gate 603 to the current source 601 to pre-charge the capacitor 605 to a negative value (such that I_{c}<0), while leaving the switch 606 open.
2. Connect the switch/gate 603 to the nanopore sequencing unit cell 602 to measure the nanopore signal (Iₚₒᵣₑ), while leaving the switch 606 open.
3. As a result of the feedback loop created by the capacitor 605, the measured signal I, which is fed to the ADC 604, may be affected by the capacitor 605, i.e., I=I_{c}+Iₚₒᵣₑ, such that I only contains the AC part of the nanopore signal.
4. Digitize the measured signal I. Since I_{c} is a known value, the current through the nanopore in the nanopore sequencing unit cell 602 can be determined by Iₚₒᵣₑ=I−I_{c}.

To release the charge stored in the capacitor 605, the switch 606 can be closed, short-circuiting the capacitor 605.

### Example Sequencing Systems and Methods

In one aspect, the disclosed technology relates to a device for sequencing polynucleotides, comprising: a sequencing cell comprising a nanopore for sensing a polynucleotide; an electronic circuit configured to measure an electrical response in the sequencing cell, the electronic circuit comprising a feedback capacitor; and a current source operably connected to the electronic circuit via a gate.

In some embodiments, the gate is configured to connect the electronic circuit to either the current source or the sequencing cell.

In some embodiments, the electronic circuit further comprises a switch connected in parallel with the feedback capacitor.

In some embodiments, the electronic circuit comprises an analog-to-digital converter.

In some embodiments, the nanopore is an opening in a protein or nucleic acid structure deposited in a lipid or polymer membrane, or wherein the nanopore is an opening in a solid-state structure.

In some embodiments, the electrical response in the sequencing cell is modulated by: nucleotides in the polynucleotide near the sensing zone of the nanopore, labels on nucleotides in the polynucleotide near the sensing zone of the nanopore, nucleotides being incorporated to the polynucleotide, labels on nucleotides being incorporated to the polynucleotide, or any combination thereof.

In some embodiments, the electrical response is an ionic current through the nanopore in the sequencing cell or equivalents thereof.

In another aspect, the disclosed technology relates to a method for sequencing polynucleotides, comprising: providing a polynucleotide to a sequencing cell comprising a nanopore; charging a feedback capacitor of an electronic circuit; and measuring an electrical response in the sequencing cell while the electronic circuit is connected to the sequencing cell such that the measured electrical response is offset by the charged feedback capacitor, wherein the electrical response depends on the identity of one or more nucleotides of polynucleotide within or near the nanopore.

In some embodiments, charging the feedback capacitor of the electronic circuit is by way of connecting the feedback capacitor to a current source via a gate.

In some embodiments, the electronic circuit is connected to the sequencing cell by way of switching the gate.

In some embodiments, the feedback capacitor is disconnected from the current source while the electronic circuit is connected to the sequencing cell.

In some embodiments, the method further includes setting a switch in parallel with the feedback capacitor to a disconnected state prior to charging the feedback capacitor.

In some embodiments, the electrical response measured by the electronic circuit is an ionic current through the nanopore or equivalents thereof.

In some embodiments, the electrical response is modulated by: nucleotides in the polynucleotide near the sensing zone of the nanopore, labels on nucleotides in the polynucleotide near the sensing zone of the nanopore, nucleotides being incorporated to the polynucleotide, labels on nucleotides being incorporated to the polynucleotide, or any combination thereof.

In some embodiments, the method further includes providing an electrolyte to the sequencing cell prior to providing the polynucleotide.

In yet another aspect, the disclosed technology relates to a system for sequencing polynucleotides, comprising: a common cis well associated with a common cis electrode; and a plurality of sequencing units comprising an electrolyte, each of the plurality of sequencing units comprising: a trans well associated with a trans electrode; a sequencing cell comprising a nanopore for sensing a polynucleotide, the nanopore fluidically connecting the trans well to the common cis well; and an electronic circuit configured to measure an electrical response in the sequencing cell, the electronic circuit comprising a feedback capacitor.

In some embodiments, for at least some of the plurality of sequencing units, the electronic circuit is operably connected to a current source via a gate.

In some embodiments, for at least some of the plurality of sequencing units, the gate is configured to connect the electronic circuit to either the current source or the sequencing cell.

In some embodiments, for at least some of the plurality of sequencing units, the electronic circuit comprises an analog-to-digital converter.

In some embodiments, for at least some of the plurality of sequencing units, the feedback capacitor is connected to an input terminal and an output terminal of the analog-to-digital converter.

### Additional Notes

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

It is to be understood that the ranges provided herein include the stated range and any value or sub-range within the stated range, as if such value or sub-range were explicitly recited. For example, a range from about 2 nm to about 20 nm should be interpreted to include not only the explicitly recited limits of from about 2 nm to about 20 nm, but also to include individual values, such as about 3.5 nm, about 8 nm, about 18.2 nm, etc., and sub-ranges, such as from about 5 nm to about 10 nm, etc. Furthermore, when “about” and/or “substantially” are/is utilized to describe a value, this is meant to encompass minor variations (up to +/−10%) from the stated value.

While several examples have been described in detail, it is to be understood that the disclosed examples may be modified. Therefore, the foregoing description is to be considered non-limiting.

While certain examples have been described, these examples have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure.

Features, materials, characteristics, or groups described in conjunction with a particular aspect, or example are to be understood to be applicable to any other aspect or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing examples. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations, one or more features from a claimed combination can, in some cases, be excised from the combination, and the combination may be claimed as a sub-combination or variation of a sub-combination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some examples, the actual steps taken in the processes illustrated and/or disclosed may differ from those shown in the figures. Depending on the example, certain of the steps described above may be removed or others may be added. Furthermore, the features and attributes of the specific examples disclosed above may be combined in different ways to form additional examples, all of which fall within the scope of the present disclosure. Also, the separation of various system components in the implementations described above should not be understood as requiring such separation in all implementations, and it should be understood that the described components and systems can generally be integrated together in a single product or packaged into multiple products. For example, any of the components for an energy storage system described herein can be provided separately, or integrated together (e.g., packaged together, or attached together) to form an energy storage system.

For purposes of this disclosure, certain aspects, advantages, and novel features are described herein. Not necessarily all such advantages may be achieved in accordance with any particular example. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain examples include, while other examples do not include, certain features, elements, and/or steps. Thus, such conditional language is not generally intended to imply that features, elements, and/or steps are in any way required for one or more examples or that one or more examples necessarily include logic for deciding, with or without user input or prompting, whether these features, elements, and/or steps are included or are to be performed in any particular example.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately,” “about,” “generally,” and “substantially” represent a value, amount, or characteristic close to the stated value, amount, or characteristic that still performs a desired function or achieves a desired result.

The scope of the present disclosure is not intended to be limited by the specific disclosures of preferred examples in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A device for sequencing polynucleotides, comprising:
a sequencing cell comprising a nanopore for sensing a polynucleotide;
an electronic circuit configured to measure an electrical response in the sequencing cell, the electronic circuit comprising a feedback capacitor; and
a current source operably connected to the electronic circuit via a gate.

2. The device of claim 1, wherein the gate is configured to connect the electronic circuit to either the current source or the sequencing cell.

3. The device of claim 1, wherein the electronic circuit further comprises a switch connected in parallel with the feedback capacitor.

4. The device of claim 1, wherein the electronic circuit comprises an analog-to-digital converter.

5. The device of claim 1, wherein the nanopore is an opening in a protein or nucleic acid structure deposited in a lipid or polymer membrane, or wherein the nanopore is an opening in a solid-state structure.

6. The device of claim 1, wherein the electrical response in the sequencing cell is modulated by: nucleotides in the polynucleotide near a sensing zone of the nanopore, labels on nucleotides in the polynucleotide near the sensing zone of the nanopore, nucleotides being incorporated to the polynucleotide, labels on nucleotides being incorporated to the polynucleotide, or any combination thereof.

7. The device of claim 1, wherein the electrical response is an ionic current through the nanopore in the sequencing cell or equivalents thereof.

8. A method for sequencing polynucleotides, comprising:
providing a polynucleotide to a sequencing cell comprising a nanopore;
charging a feedback capacitor of an electronic circuit; and
measuring an electrical response in the sequencing cell while the electronic circuit is connected to the sequencing cell such that the measured electrical response is offset by the charged feedback capacitor, wherein the electrical response depends on identity of one or more nucleotides of polynucleotide within or near the nanopore.

9. The method of claim 8, wherein charging the feedback capacitor of the electronic circuit is by way of connecting the feedback capacitor to a current source via a gate.

10. The method of claim 9, wherein the electronic circuit is connected to the sequencing cell by way of switching the gate.

11. The method of claim 9, wherein the feedback capacitor is disconnected from the current source while the electronic circuit is connected to the sequencing cell.

12. The method of claim 8, further comprising setting a switch in parallel with the feedback capacitor to a disconnected state prior to charging the feedback capacitor.

13. The method of claim 8, wherein the electrical response measured by the electronic circuit is an ionic current through the nanopore or equivalents thereof.

14. The method of claim 8, wherein the electrical response is modulated by: nucleotides in the polynucleotide near a sensing zone of the nanopore, labels on nucleotides in the polynucleotide near the sensing zone of the nanopore, nucleotides being incorporated to the polynucleotide, labels on nucleotides being incorporated to the polynucleotide, or any combination thereof.

15. The method of claim 8, further comprising providing an electrolyte to the sequencing cell prior to providing the polynucleotide.

16. A system for sequencing polynucleotides, comprising:
a common cis well associated with a common cis electrode; and
a plurality of sequencing units comprising an electrolyte, each of the plurality of sequencing units comprising:
a trans well associated with a trans electrode;
a sequencing cell comprising a nanopore for sensing a polynucleotide, the nanopore fluidically connecting the trans well to the common cis well; and
an electronic circuit configured to measure an electrical response in the sequencing cell, the electronic circuit comprising a feedback capacitor.

17. The system of claim 16, wherein, for at least some of the plurality of sequencing units, the electronic circuit is operably connected to a current source via a gate.

18. The system of claim 17, wherein, for at least some of the plurality of sequencing units, the gate is configured to connect the electronic circuit to either the current source or the sequencing cell.

19. The system of claim 16, wherein, for at least some of the plurality of sequencing units, the electronic circuit comprises an analog-to-digital converter.

20. The system of claim 19, wherein, for at least some of the plurality of sequencing units, the feedback capacitor is connected to an input terminal and an output terminal of the analog-to-digital converter.


SEQUENCE LISTING (1 sequence)

SEQ ID NO: 1 - DNA, 22 bp, synthetic construct
  misc_feature 1..22: note = DNA 110
  source 1..22: mol_type = other DNA
agccacctttttaccaagccag